(19) United States
(12) Patent Application Publication
(10) Pub. No.: US 2023/0016081 A1
(43) Pub. Date: Jan. 19, 2023

(54) MULTI-FUNCTIONAL AIR PURIFICATION FILTER AND PURIFICATION DEVICE INCLUDING THE SAME

(71) Applicant: SAMSUNG ELECTRONICS CO., LTD., Suwon-si (KR)

(72) Inventors: Sungwon KIM, Suwon-si (KR); Saemi KIM, Suwon-si (KR); Soomin PARK, Suwon-si (KR); Heejin PARK, Suwon-si (KR); Changbae LIM, Suwon-si (KR); Yeonkyoung JUNG, Suwon-si (KR); Yongwon JEONG, Suwon-si (KR); Sungpil HONG, Suwon-si (KR)

(73) Assignee: SAMSUNG ELECTRONICS CO., LTD., Suwon-si (KR)

(21) Appl. No.: 17/745,380

(22) Filed: May 16, 2022

Related U.S. Application Data
(63) Continuation of application No. PCT/KR2022/004522, filed on Mar. 30, 2022.

(30) Foreign Application Priority Data
Jul. 13, 2021 (KR) 10-2021-0091453

(51) Int. Cl. B01D 46/00; B01D 53/00; B01D 53/88; B01D 46/52; B01D 39/16; A61L 9/20

(52) U.S. Cl. CPC B01D 46/0028; B01D 46/0036; B01D 53/007; B01D 53/885; B01D 46/521; B01D 39/1623; A61L 9/205; B01D 2253/108; B01D 2253/106; B01D 2253/11; B01D 2253/102; B01D 2259/804; B01D 2255/802; B01D 2255/20707; B01D 2255/20776; B01D 2255/20715; B01D 2255/20723; B01D 2239/0407; B01D 2239/0442; B01D 2239/0618; B01D 2239/0654; A61L 2209/14; A61L 2209/22; A61L 2209/21

(57) ABSTRACT

A multi-functional air purification filter includes: a breathable support layer; a mesh layer provided on the breathable support layer; and a filter layer provided on the mesh layer, wherein at least a portion of the multi-functional air purification filter has a plurality of bends, and the mesh layer includes a coating layer of at least one of a photocatalytic material, an adsorption material, or a sterilizing material.

### CROSS-REFERENCE TO RELATED APPLICATIONS

This application is a continuation of International Application No. PCT/KR2022/004522, filed on Mar. 30, 2022, in the Korean Intellectual Property Receiving Office and claiming priority to Korean Patent Application No. 10-2021-0091453, filed on Jul. 13, 2021, in the Korean Intellectual Property Office, the disclosures of which are incorporated by reference herein in their entireties.

### BACKGROUND

### 1. Field

The disclosure relates to a multi-functional air purification filter, e.g., a multi-functional air purification filter, and a purification device including the same.

### 2. Description of Related Art

Various products for managing hygiene and maintaining cleanliness are increasingly being developed and utilized due to environmental degradation, such as air contamination by high-concentration fine dust and/or yellow sand or water contamination by various wastes and chemicals, and infectious diseases including bacteria and viruses. For example, purification devices for purifying indoor air or drinking water, such as air purifiers or water purifiers, have appeared, and the use of purification devices is increasing in everyday life.

The purification device may collect, adsorb, or remove pollutant particles by including a non-woven fabric filter or an electrostatic filter, and/or it may remove bacteria and/or viruses by including an activated carbon or metal oxide filter. For example, while air or drinking water passes through the filter, contaminants, various bacteria and/or viruses may be removed by the filter. In general, purification devices may be installed in the user's living/office space or various living facilities where a large number of people come and go.

A composite filter disposed in a purification device is to implement multiple performances by arranging filters having different functions in parallel but this increases the volume and weight of the purification device, and reduces the air flow of the purification device due to an increase in differential pressure that occurs each time an individual-filter is added. As a result, the performance (and area of application) of the purification device is reduced.

### SUMMARY

One or more embodiments may provide a multi-functional air purification filter with multiple performances.

Further, one or more embodiments may facilitate the design of a purification device, such as an air purifier, due to a reduction in the relative volume of the multi-functional air purification filter.

Further still, one or more embodiments may increase the application area of the purification device due to a decrease in the relative differential pressure of the multi-functional air purification filter.

According to an aspect of the disclosure, a multi-functional air purification filter includes: a breathable support layer; a mesh layer provided on the breathable support layer; and a filter layer provided on the mesh layer, wherein at least a portion of the multi-functional air purification filter has a plurality of bends, and the mesh layer includes a coating layer of at least one of a photocatalytic material, an adsorption material, or a sterilizing material.

The photocatalytic material may include at least one of titanium dioxide (TiO₂), tungsten oxide (WO₃), zirconium oxide (ZrO₂), zinc oxide (ZnO), cadmium sulfide (CdS), or vanadium oxide (V₂O₃).

The adsorption material may include at least one of zeolite, sepiolite, mesoporous silica (mesoporous SiO₂), silica (SiO₂), activated carbon, or clay.

The sterilizing material may include at least one of Cu, a Cu compound, Ag, an Ag compound, Zn, or a Zn compound.

A ratio of the adsorption material to the photocatalytic material may be 1.0 wt % to 80.0 wt %.

A ratio of the sterilizing material to the photocatalytic material may be 0.1 wt % to 10.0 wt %.

The breathable support layer may include at least one of nonwoven fabric, felt, polyethylene terephthalate (PET), or polypropylene (PP).

The mesh layer may include at least one of polyethylene terephthalate (PET), polypropylene (PP), polyethylene (PE), polytetrafluoroethylene (PTFE), stainless use steel (SUS), Ti, Al, or Cu.

The filter layer may include at least one of polyethylene terephthalate (PET), polypropylene (PP), polyethylene (PE), or polytetrafluoroethylene (PTFE).

The multi-functional air purification filter may further include beads filling empty spaces provided by the plurality of bends of the multi-functional air purification filter, the beads including containing a photocatalytic material, an adsorption material, and a sterilizing material.

According to an aspect of the disclosure, a purification device includes: a multi-functional air purification filter including a breathable support layer, a mesh layer provided on the breathable support layer, and a filter layer provided the mesh layer, wherein at least a portion of the multi-functional air purification filter has a plurality of bends, and the mesh layer includes a coating layer of at least one of a photocatalytic material, an adsorption material, or a sterilizing material.

The purification device may further include: a housing in which the multi-functional air purification filter is provided; and a blowing fan provided in the housing and configured to introduce external air through the multi-functional air purification filter to an inside of the housing.

The purification device may further include a light source configured to irradiate the multi-functional air purification filter with light.

The photocatalytic material may include at least one of titanium dioxide (TiO₂), tungsten oxide (WO₃), zirconium oxide (ZrO₂), zinc oxide (ZnO), cadmium sulfide (CdS), or vanadium oxide (V₂O₃), the adsorption material may include at least one of zeolite, sepiolite, mesoporous silica (mesoporous SiO₂), silica (SiO₂), activated carbon, or clay, and the sterilizing material may include at least one of Cu, a Cu compound, Ag, an Ag compound, Zn, or a Zn compound.

A ratio of the adsorption material to the photocatalytic material is 1.0 wt % to 80.0 wt %.

A ratio of the sterilizing material to the photocatalytic material is 0.1 wt % to 10.0 wt %.

The breathable support layer may include at least one of nonwoven fabric, felt, polyethylene terephthalate (PET), or polypropylene (PP).

The mesh layer may include at least one of polyethylene terephthalate (PET), polypropylene (PP), polyethylene (PE), polytetrafluoroethylene (PTFE), stainless use steel (SUS), Ti, Al, or Cu.

The filter layer may include at least one of polyethylene terephthalate (PET), polypropylene (PP), polyethylene (PE), or polytetrafluoroethylene (PTFE).

According to an aspect of one or more embodiments, there may be provided a multi-functional air purification filter having multiple performances or a purification device including the same.

According to an aspect of one or more embodiments, as compared with a configuration in which a plurality of filters are arranged, a relative reduction in the volume of the multi-functional air purification filter is achieved, allowing the purification device, e.g., an air purifier, to be made compact, and enhancing the degree of freedom in design in purification devices with the same size.

According to an aspect of one or more various embodiments, as compared with a configuration in which a plurality of filters are arranged, a relative reduction in the differential pressure of the multi-functional air purification filter is obtained, increasing the application area of the purification device.

### BRIEF DESCRIPTION OF THE DRAWINGS

The above and other aspects, features, and advantages of certain embodiments of the present disclosure will be more apparent from the following description taken in conjunction with the accompanying drawings, in which:

FIGS. 1A, 1B, and 1C are views illustrating multi-functional air purification filters according to various embodiments;

FIGS. 2A and 2B are views illustrating a multi-functional air purification filter according to various embodiments;

FIG. 3 is a view illustrating an air purifier, as an example of a purification device having a multi-functional air purification filter applied thereto, according to various embodiments;

FIG. 4 is an exploded perspective view illustrating the purification device of FIGS. 3; and

FIGS. 5A and 5B are views illustrating a manufacturing device of a multi-functional air purification filter according to various embodiments.

### DETAILED DESCRIPTION

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements.

It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. For example, the expression, “at least one of A, B, and (or) C,” should be understood as including only A, only B, only C, both A and B, both A and C, both B and C, or all of A, B, and C. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., via a wired coupling), wirelessly, or via a third element.

According to various embodiments, each component of the above-described components may include a single entity or multiple entities. Some of the plurality of entities may be separately disposed in different components. According to various embodiments, one or more of the above-described components may be omitted, or one or more other components may be added. Alternatively or additionally, a plurality of components may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

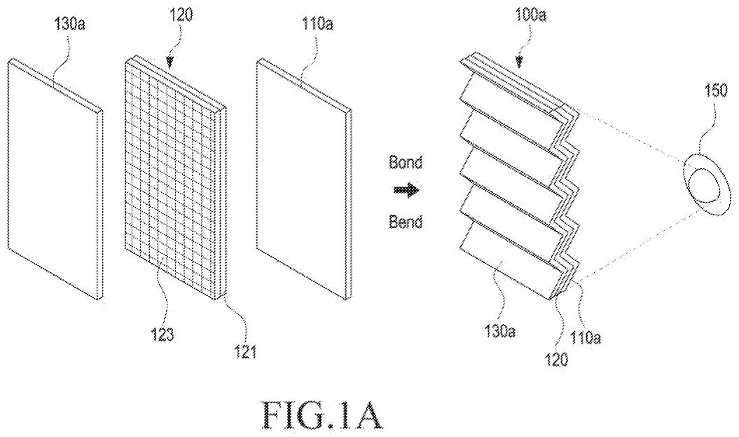

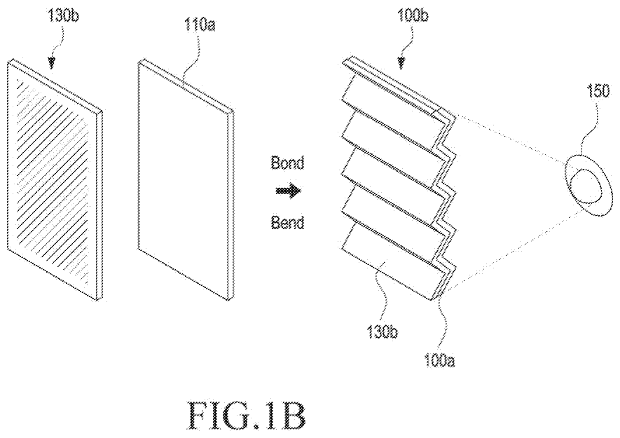

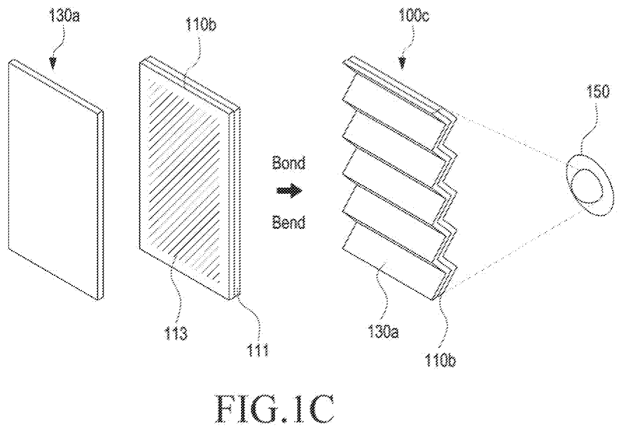

FIGS. 1A, 1B, and 1C are views illustrating multi-functional air purification filters according to various embodiments.

Referring to FIG. 1A, a multi-functional air purification filter 100a may include a support layer 110a, a mesh layer 120 disposed on a surface of the support layer 110a, and a filter layer 130a disposed on a surface of the mesh layer 120.

According to various embodiments, the support layer 110a is a layer that substantially determines the shape of the multi-functional air purification filter 100a and may be formed of a material including at least one of a breathable mesh, non-woven fabric, felt, polyethylene terephthalate (PET), or polypropylene (PP). According to an embodiment, the support layer 110a may have a shape in which at least a portion thereof is bent multiple times. For example, the support layer 110a may have a shape with ridges and valleys periodically repeated as it is bent in a zig-zag manner. According to an embodiment, the support layer 110a may include a polymer or metal material and collect contaminants using static electricity according to the included material. According to an embodiment, the support layer 110a may have a light transmittance of 50% to 58%.

According to various embodiments, the mesh layer 120 may be coupled, coated, or attached (an adhesive may be used) to a surface of the support layer 110a. The mesh layer 120 may be formed in a mesh shape with dense pores.

According to various embodiments, the mesh layer 120 may be formed of a polymer and/or metal material. According to an embodiment, the mesh layer 120 may be formed of a polymer (plastic) material (polyethylene terephthalate (PET), polypropylene (PP), polyethylene (PE), or polytetrafluoroethylene (PTFE)). In another embodiment, the mesh layer 120 may be formed of a metal material (stainless use steel (SUS), Ti, Al, Cu, or various alloys). In another embodiment, the mesh layer 120 may be formed of a composite or hybrid material of metal, ceramic, and polymer. The mesh layer 120 may be formed of a porous mesh material.

According to various embodiments, the filter layer 130a may be formed of a polymer (plastic) material (PET, PP, PE, or PTFE).

According to various embodiments, the filter layer 130a may be coupled, coated, or attached (an adhesive may be used) to a surface of the mesh layer 120. According to an embodiment, the filter layer 130a may be formed by a melt blown process in which a thermoplastic polymer is fused and extruded through a nozzle. According to an embodiment, the filter layer 130a may have a light transmittance of 12% to 15%.

According to various embodiments, the support layer 110a and the mesh layer 120 are exemplified as components different from the filter layer 130a, but this is for convenience of description, and the support layer 110a and the mesh layer 120 may function as an additional filter in combination with the filter layer 130a. For example, the filter layer 130a may provide a filtering function for removing fine dust, and the support layer 110a and/or the mesh layer 120 may function as a filter to remove contaminants having particles larger than fine dust. For example, the support layer 110a and/or the mesh layer 120 may adsorb or collect dust or organic compounds floating in the air.

According to various embodiments, to provide sterilization and gas adsorption/decomposition functions, at least one of the support layer 110a, the mesh layer 120 or the filter layer 130a may include a hybrid material, and the hybrid material may include a composite of a photocatalytic material and an adsorption material (and bactericidal material).

According to various embodiments, each or at least one of the support layer 110a, the mesh layer 120, and the filter layer 130a may be composed of a plurality of layers, and each or at least one of the support layer 110a, the mesh layer 120 or the filter layer 130a, or one of the plurality of layers may include a hybrid material.

According to various embodiments, the photocatalytic material may include at least one of titanium dioxide (TiO₂) (in the form of anatase, rutile, or anatase+rutile), tungsten oxide (WO₃), zirconium oxide (ZrO₂), zinc oxide (ZnO), cadmium sulfide (CdS), or vanadium oxide (V₂O₃).

According to various embodiments, the adsorption material may include at least one of zeolite, sepiolite, mesoporous silica (mesoporous SiO₂), silica (SiO₂), activated carbon, or clay. Zeolite may include natural zeolite or synthetic zeolite, and the synthetic zeolite may include A zeolite, X zeolite, Y zeolite, zeolite socony mobile number 5 (ZSM-5) zeolite, or beta zeolite.

According to various embodiments, the ratio of the adsorption material to the photocatalytic material may be 1.0-80.0 wt % or 5-50 wt %.

According to various embodiments, the sterilizing material may include Cu or its compound (CuO, Cu₂O, CuS, CuCl₂, CuSO₄, Cu(NO₃)₂, Cu₂(OH)₃ etc.), or Ag or its compound (AgO, AgNO₃, Ag₂SO₄ etc.), or Zn or its compound (ZnO, ZnO₂ etc.). The sterilizing material may include a material (e.g., Cu, CuO, Cu₂O, Ag, ZnO) that may react with the photocatalytic material to secure visible light sensitivity among the sterilizing materials.

According to various embodiments, the ratio of the sterilizing material to the photocatalytic material may be 0.1-10.0 wt % or 0.1-3 wt %.

According to various embodiments, for adding the hybrid material, at least one of the support layer 110a, the mesh layer 120 or the filter layer 130a may contain a hybrid material (or is formed of a material containing a hybrid material) or may include a coating layer of a hybrid material. According to an embodiment, the mesh layer 120 may include a body 121 having a mesh structure (hereinafter, the body may be referred to as a mesh body) and a coating layer 123 which includes a photocatalyst material and an adsorption material (and a sterilizing material) and is disposed on a surface of the body 121 or formed on the surface of the mesh structure. In another embodiment, the mesh layer 120 may be formed of a hybrid material including a photocatalytic material and an adsorption material (and a sterilizing material). For example, the mesh structure of the body 121 may be formed of a material to which a photocatalytic material and an adsorption material (and a sterilizing material) are added. The body 121 and the coating layer 123 may be arranged in the order as shown in FIG. 1A or in the opposite order.

According to various embodiments, a solution in which the coating material is dispersed may be sprayed onto the body 121, or the body 121 may be dipped in a solution in which the coating material is dispersed, followed by thermal treatment (stabilizing the coating layer) according to the mesh material, thereby forming the coating layer 123.

According to various embodiments, the support layer 110a, the mesh layer 120, and the filter layer 130a may be coupled to each other, and may be formed into a preset shape. According to an embodiment, the multi-functional air purification filter 100a including the support layer 110a, the mesh layer 120 and the filter layer 130a may have a shape in which at least a portion is bent a plurality of times. For example, the multi-functional air purification filter 100a may have a shape with ridges and valleys periodically repeated as it is bent in a zig-zag manner.

According to various embodiments, the light source 150 may be positioned on one side of the multi-functional air purification filter 100a and may radiate light toward the multi-functional air purification filter 100a. The photocatalytic material of the multi-functional air purification filter 100a may react with light radiated from the light source 150 to remove harmful gases, odor substances, microorganisms, etc. Here, the ‘photocatalyst material reacts with light to remove harmful gases, odor substances, microorganisms, etc.’ means that the photocatalytic material is exposed to light to reduce or deoxidize the adsorption material or sterilizing material, thereby maintaining or enhancing the capability of removing harmful gases, odor substances, microorganisms, etc. The light source 150 may output light suitable for causing a photocatalytic reaction in the photocatalytic material included in the multi-functional air purification filter 100a. For example, the light source 150 may be implemented as a device, such as a fluorescent lamp or an incandescent lamp or a light emitting diode (LED), and it may output at least one type of light among white light, red light, green light, blue light, ultraviolet (UV) light, visible light, or infrared light. The photocatalytic material included in the air purification filter 100a may react with the light radiated from the light source 150 to purify the air. The photocatalytic material may completely decompose volatile organic compounds (VOCs) into carbon dioxide and water, which are harmless to the human body, and may also be effective in removing bacteria or microorganisms when UV light is used. For example, the photocatalytic material may include titanium dioxide (TiO₂). Titanium dioxide generates radicals (e.g., OH) when exposed to ultraviolet rays. The strong oxidizing power of these radicals may sterilize microorganisms and decompose odor-causing substances. The photocatalytic material may decompose pollutants adsorbed in the filter, using the light source 150, so that it may be used semi-permanently. Therefore, it is also beneficial in terms of reduction in maintenance cost due to filter replacement and ease of management. For example, the air purification filter 100a may remove harmful substances, such as nitrogen oxides (NOx), sulfur oxides (SOx), formaldehyde, and the like in the air (air purification). Further, the air purification filter 100a may adsorb and/or decompose odors (deodorization), such as acetaldehyde, ammonia, and hydrogen sulfide, and may sterilize various viruses, pathogens and bacteria, prevent decay (antibacterial action), and decompose organic substances, such as cigarette smoke and oil residue (antifouling action).

According to various embodiments, the air purification filter 100a provides sterilization and gas adsorption/decomposition functions and thus may provide multiple performances.

According to various embodiments, the support layer 110a is a layer in which relatively large dust and bacteria are adsorbed well, and which contains an Ag- or Zn-based sterilizing material having excellent bacterial sterilization performance, as the sterilizing material in the hybrid material, thereby strengthening the bacteria sterilizing performance. According to an embodiment, the Ag- or Zn-based sterilizing material may be included in a 0.1 to 10.0 wt % proportion of the support layer 110a.

According to various embodiments, the mesh layer 120 is a layer that performs functions (deodorization, decomposition, sterilization, etc.) other than dust removal, and may be coated with a photocatalytic material and an adsorption material in excessive quantities. According to an embodiment, the photocatalytic material and the adsorption material may be included in a 1 to 50 wt % proportion of the mesh layer 120.

According to various embodiments, the filter layer 130a is a layer in which relatively small ultrafine dust, viruses, etc. are adsorbed well, and as a sterilizing material in the hybrid material, it may include a Cu-based sterilizing material having excellent virus sterilization performance and thus strengthen the virucidal performance. According to an embodiment, the sterilizing material may be included in a 0.1 to 5 wt % proportion of the filter layer 130a.

According to various embodiments, since the air purification filter 100a has a reduced volume as compared to a configuration in which a plurality of filters are disposed, the purification device including the air purification filter 100a may reduce in size, and the freedom of design for purification devices with the same size may be enhanced.

According to various embodiments, the air purification filter 100a may include a hybrid material corresponding to the individual filters, thereby reducing the differential pressure as compared to a configuration in which a plurality of filters are disposed.

According to various embodiments, the air purification filter 100a may include the mesh layer 120 having regular pores, thereby reducing pressure loss.

According to various embodiments, the air purification filter 100a may include the conductive mesh layer 120, such as of metal, thereby providing an additional performance enhancement technology through the use of a heat transfer function, voltage application, or the like.

According to various embodiments, the air purification filter 100a may include the support layer 110a having a shape with multiple bends, increasing the surface area and hence maximizing the purification performance. Further, it may contain an excess of hybrid material in the same volume.

According to various embodiments, the air purification filter 100a covers the coating layer 123 of the mesh layer 120 with the support layer 110a and the filter layer 130a, preventing dust caused by the peel-off of the coating layer 123.

According to various embodiments, the air purification filter 100a forms the coating layer 123 through coating according to the shape of the mesh layer 120, so that the content of the hybrid material and the differential pressure may be adjusted, as desired, depending on the shape of the mesh layer 120.

According to various embodiments, in the air purification filter 100a, the support layer 110a and the filter layer 130a may function as protective films of the mesh layer 120, thereby preventing a reduction in the performance of the mesh layer 120 due to external dust.

According to various embodiments, the air purification filter 100a may secure the maximum performance and implement low pressure loss by maximizing the content of the hybrid material.

Hereinafter, repeated description of components denoted by the same/similar reference numbers to those of the components of FIG. 1A will be omitted.

Referring to FIG. 1B, a multi-functional air purification filter 100b may include a support layer 110a and a filter layer 130b disposed on a surface of the support layer 110a.

According to various embodiments, the support layer 110a is a layer that substantially determines the shape of the multi-functional air purification filter 100b and may be formed of at least one of a breathable mesh, a non-woven fabric, or felt.

According to various embodiments, the filter layer 130b may be coupled, coated, or attached to a surface of the support layer 110a. According to an embodiment, the filter layer 130b may be formed by a melt blown process in which a thermoplastic polymer is fused and extruded through a nozzle.

According to various embodiments, for adding the hybrid material, at least one of the support layer 110a or the filter layer 130b may contain a hybrid material (or is formed of a material containing a hybrid material) or may include a coating layer of a hybrid material. According to an embodiment, the filter layer 130b may be formed of a hybrid material including a photocatalytic material and an adsorption material (and a sterilizing material). In another embodiment, the filter layer 130b may include a flat body and a coating layer which includes a photocatalytic material and an adsorption material (and a sterilizing material) and is disposed on a surface of the body.

According to various embodiments, a coating material (or hybrid material) may be dispersed in a solution to be melt blown. The filter layer 130b may be formed by melt-blowing the solution in which the coating material is dispersed.

According to various embodiments, the support layer 110a and the filter layer 130a may be coupled to each other, and may be formed into a preset shape. According to an embodiment, the multi-functional air purification filter 100b including the support layer 110a and the filter layer 130b may have a shape in which at least a portion is bent a plurality of times. For example, the multi-functional air purification filter 100b may have a shape with ridges and valleys periodically repeated as it is bent in a zig-zag manner.

According to various embodiments, the light source 150 may be positioned on one side of the multi-functional air purification filter 100b and may radiate light toward the multi-functional air purification filter 100b. The photocatalytic material of the multi-functional air purification filter 100b may react with light radiated from the light source 150 to remove harmful gases, odor substances, microorganisms, etc.

According to various embodiments, the air purification filter 100b contains a hybrid material in the melt-blown solution. Thus, the air purification filter 100b may obtain a pressure loss similar to that of the conventional melt-blown layer. Further, since the existing process may be used as it is, only with the melt-blown solution changed, the air purification filter 100b may be easy to mass-produce.

Referring to FIG. 1C, a multi-functional air purification filter 100c may include a support layer 110b and a filter layer 130a disposed on a surface of the support layer 110b.

According to various embodiments, the support layer 110b is a layer that substantially determines the shape of the multi-functional air purification filter 100c and may be formed of at least one of a breathable mesh, a non-woven fabric, or felt.

According to various embodiments, the filter layer 130a may be coupled, coated, or attached to a surface of the support layer 110b. The filter layer 130a may be formed by a melt blown process in which a thermoplastic polymer is fused and extruded through a nozzle.

According to various embodiments, for adding the hybrid material, at least one of the support layer 110b or the filter layer 130a may contain a hybrid material (or is formed of a material containing a hybrid material) or may include a coating layer of a hybrid material. According to an embodiment, the support layer 110b may include a flat body 111 and a coating layer 113 which includes a photocatalytic material and an adsorption material (and a sterilizing material) and is disposed on a surface of the body 111. In another embodiment, the support layer 110b may be formed of a hybrid material including a photocatalytic material, an adsorption material, and a sterilizing material. The body 111 and the coating layer 113 may be arranged in the order as shown in FIG. 1C or in the opposite order.

According to various embodiments, a solution in which the coating material is dispersed may be sprayed onto the body 111, or the body 111 may be dipped in a solution in which the coating material is dispersed, followed by thermal treatment (stabilizing the coating layer) according to the material, thereby forming the coating layer 113.

According to various embodiments, the coating material may be dispersed in a solution that mainly forms the support layer 110b, and the support layer 110b may be formed using the solution in which the coating material is dispersed.

According to various embodiments, the support layer 110b and the filter layer 130a may be coupled to each other, and may be formed into a preset shape. According to an embodiment, the multi-functional air purification filter 100c including the support layer 110b and the filter layer 130a may have a shape in which at least a portion is bent a plurality of times. For example, the multi-functional air purification filter 100c may have a shape with ridges and valleys periodically repeated as it is bent in a zig-zag manner.

According to various embodiments, the light source 150 may be positioned on one side of the multi-functional air purification filter 100c and may radiate light toward the multi-functional air purification filter 100c. The photocatalytic material of the multi-functional air purification filter 100c may react with light radiated from the light source 150 to remove harmful gases, odor substances, microorganisms, etc.

According to various embodiments, as the support layer 110b of the air purification filter 100c is formed of a hybrid material including a photocatalyst material, an adsorption material, and a sterilizing material, it may maintain a pressure loss similar to that of a conventional support layer.

According to various embodiments, the air purification filter 100c adds a simple coating process or may use the existing process simply with the support layer formation layer changed and may thus be easy to mass-produce.

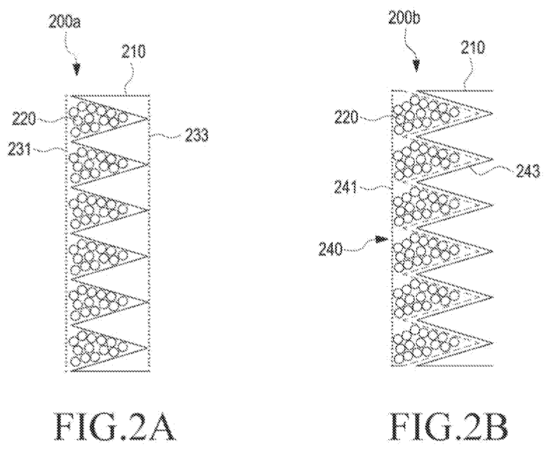

FIGS. 2A and 2B are views illustrating a multi-functional air purification filter according to various embodiments.

Referring to FIG. 2A, a multi-functional air purification filter 200a may include a HEPA filter 210, beads 220 filling an empty space provided by a shape with a plurality of bends of the HEPA filter 210, a first mesh case 231 disposed on the front surface of the HEPA filter 210 providing the empty space, and a second mesh case 233 disposed on the rear surface of the HEPA filter 210.

According to various embodiments, the HEPA filter 210 is a layer that substantially determines the shape of the multi-functional air purification filter 200a and may be formed of at least one of a breathable mesh, a non-woven fabric, or felt. According to an embodiment, the HEPA filter 210 may be a HEPA filter of a non-hybrid material (or a hybrid material) having a configuration including a support layer, a mesh layer and a filter layer, e.g., as shown in FIG. 1A. According to an embodiment, the HEPA filter 210 may be a HEPA filter of a non-hybrid material (or a hybrid material) having a configuration including a support layer and a filter layer, e.g., as shown in FIG. 1B or 1C. According to an embodiment, the HEPA filter 210 may have a shape in which at least a portion thereof is bent multiple times. For example, the HEPA filter 210 may have a shape with ridges and valleys periodically repeated as it is bent in a zig-zag manner.

According to various embodiments, the beads 220 are beads of a hybrid material and may include a photocatalytic material and an adsorption material (and a sterilizing material). According to an embodiment, the beads 220 may be formed based on (or to include) water, a photocatalytic material, e.g., titanium dioxide (TiO₂), a zeolite, and a sterilizing material. According to an embodiment, the zeolite may include a natural zeolite or a synthetic zeolite (zeolite A, zeolite X, zeolite Y, ZSM-5 zeolite, or beta zeolite). For example, the beads 220 may be formed as water and titanium dioxide (TiO₂), which is a photocatalytic material, zeolite and a sterilizing material may be mixed and granulated, sieved and dried. The shape and/or size of the beads 220 may be appropriately selected depending on the type of gas to be removed, removal rate, and/or removal rate. The shape of the beads 220 may be, e.g., a spherical shape, a cylindrical shape, a hexahedral shape, or a porous shape, and the size of the beads 220 may be, e.g., 0.5 mm to 5 mm. However, the beads are not limited to a specific shape and size, and may be formed in any shape and any size.

According to various embodiments, the beads 220 may have a smooth surface and/or may have protrusions on the surface to increase the reaction surface area.

According to various embodiments, the first mesh case 231 may be coupled, coated, or attached to the front surface of the HEPA filter 210. The first mesh case 231 may be formed in a mesh shape with dense pores.

According to various embodiments, the second mesh case 233 may be coupled, coated, or attached to the rear surface of the HEPA filter 210. The second mesh case 233 may be formed in a mesh shape with dense pores.

Referring to FIG. 2B, a multi-functional air purification filter 200b may include a HEPA filter 210, a mesh case 240 disposed on the front surface of the HEPA filter 210, and beads 220 filling an empty space provided by the shape with multiple bends of the mesh case 240.

According to various embodiments, the HEPA filter 210 is a layer that substantially determines the shape of the multi-functional air purification filter 200b and may be formed of at least one of a breathable mesh, a non-woven fabric, or felt. According to an embodiment, the HEPA filter 210 may be a HEPA filter of a non-hybrid material (or a hybrid material) having a configuration including a support layer, a mesh layer and a filter layer, e.g., as shown in FIG. 1A. According to an embodiment, the HEPA filter 210 may be a HEPA filter of a non-hybrid material (or a hybrid material) having a configuration including a support layer and a filter layer, e.g., as shown in FIG. 1B or 1C. According to an embodiment, the HEPA filter 210 may have a shape in which at least a portion thereof is bent multiple times. For example, the HEPA filter 210 may have a shape with ridges and valleys periodically repeated as it is bent in a zig-zag manner.

According to various embodiments, the mesh case 240 may include a first mesh case 241 and a second mesh case 243. The mesh case 240 may be formed in a mesh shape with dense pores. In the mesh case 240, the first mesh case 241 and the second mesh case 243 may be coupled to each other or integrally formed. The second mesh case 243 may have a shape corresponding to the shape of the HEPA filter 210 to be inserted into and coupled to the empty space of the HEPA filter 210. According to an embodiment, the second mesh case 243 may have a shape in which at least a portion thereof is bent multiple times. For example, the second mesh case 243 may have a shape with ridges and valleys periodically repeated as it is bent in a zig-zag manner. According to an embodiment, the mesh case 240 may be coupled to or separated from the HEPA filter 210.

According to various embodiments, the beads 220 are beads of a hybrid material and may include a photocatalytic material and an adsorption material (and a sterilizing material). The beads 220 may fill the inner empty space of the mesh case 240.

According to various embodiments, the multi-functional air purification filter 200a or 200b may have the total volume reduced as compared to when individual filters are arranged in parallel.

According to various embodiments, the multi-functional air purification filters 200a and 200b may adjust the increase in differential pressure by adjusting the filling amount of the beads 220.

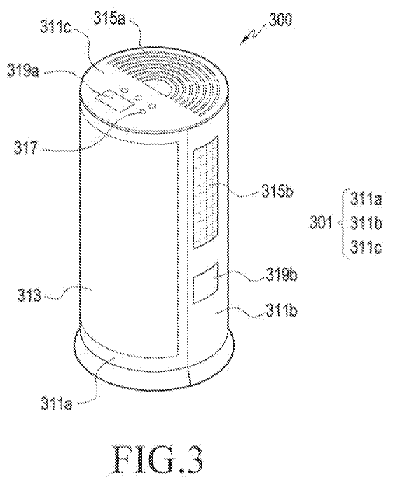

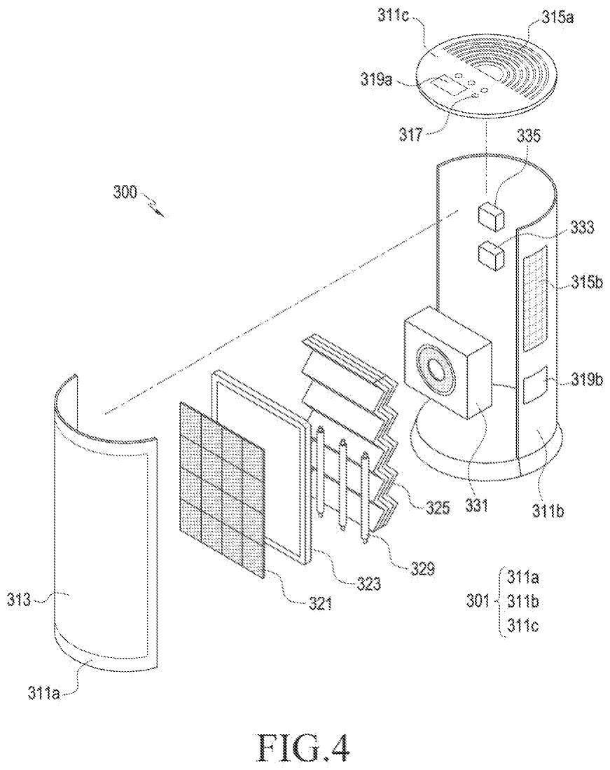

FIG. 3 is a view illustrating an air purifier, as an example of a purification device 300 having a multi-functional air purification filter (e.g., the multi-functional air purification filter 100a, 100b, or 100c of FIGS. 1A to 1C) applied thereto, according to various embodiments. FIG. 4 is an exploded perspective view illustrating the purification device 300 of FIG. 3.

Referring to FIGS. 3 and 4, according to various embodiments, a purification device 300 including a multi-functional air purification filter 325 may be, e.g., an air purifier, and may include a home appliance that is installed indoor in a building, e.g., home or office, to purify air. The air purifier may collect or remove dust or gas floating in the air, and it may remove or inactivate bacteria or viruses according to an embodiment. The purification device 300 may include a blowing fan 331 to circulate the air in the indoor space while allowing the air to pass through various filters 321, 323, and 325. According to an embodiment, the purification device 300 may further include a function of controlling the temperature and humidity of indoor air. For example, the purification device 300 may be a home appliance that includes, or selectively combine, the functions of an air purifier, an air conditioner, and/or a humidifier. In some embodiments, the multi-functional air purification filter 325 may be included in a water purifier, refrigerator, kimchi refrigerator, washing machine, dryer, clothing care device, shoe closet, closet, septic tank, and/or air conditioning system to provide deodorizing, antibacterial, and/or anti-virus functions.

According to various embodiments, the purification device 300 may include a housing 301 that forms the outer appearance and provides an inner space, an inlet 313 that is formed on one side of the housing 301 to suck in air, outlets 315a and 315b that discharge the air introduced into the inside of the housing 301 and purified, an input unit 317 for inputting user commands, and display units 319a and 319b for displaying the operation state of the purification device 300.

According to various embodiments, the housing 301 may include a main body 311b, a front cover 311a that may be coupled to the main body 311b, and an upper cover 311c. Some of the aforementioned components may be omitted, or one or more other components may be further added to the housing 301. FIG. 4 illustrates a configuration in which the front cover 311a or upper cover 311c is separated from the main body 311b, but they may be integrally formed. Other various embodiments may also be applicable.

According to various embodiments, the number and position of the inlet 313 and the outlet 315a and 315b are not limited to any particular embodiment. FIG. 3 or 4 illustrates that the inlet 313 is formed in the front cover 311a of the housing 301, and the first and second outlets 315a and 315b are in the front cover 311a and the upper cover 311c, respectively. However, embodiments are not limited thereto.

According to various embodiments, the input unit 317 may include a power button for turning on or off the purification device 300, a timer button for setting a driving time of the purification device 300, and a lock button for limiting the manipulation of the input unit to prevent wrong manipulation of the input unit. There may further be included a button for inputting various control information for the purification device 300. In this case, the input unit 317 may adopt a push switch type in which an input signal is generated by the user's pressing or a touch switch type in which an input signal is generated through the user's touch on her body portion. If the input unit 317 adopts the touch switch type, the input unit 317 may be integrally implemented with the display unit 319a.

According to various embodiments, the display units 319a and 319b may display information about the state of the purification device 300. For example, the display units 319a and 319b may display information about the degree of contamination of the filters 321, 323, and 325, information about the time of exchanging or washing the filters 321, 323, and 325, information about the state of the filters 321, 323, and 325 (e.g., accrued use days or accrued use time), and information about the current operation state (e.g., information about sensed air quality, air speed or direction). In an embodiment, such information may be provided through the display units 319a and 319b or through another electronic device (e.g., a smartphone) interworking with the purification device 300. In another embodiment, the display units 319a and 319b may be disposed in any positions on the housing 301 where it may easily be viewed by the user. FIG. 3 or 4 illustrates a display unit 319a disposed on the upper cover 311c and a display unit 319b disposed on the main body 311b as the display units 319a and 319b, but it should be noted that various embodiments are not limited to the configuration shown in the drawings.

According to various embodiments, the purification device 300 may include filters, e.g., a pre-filter 321, a HEPA filter 323, and a multi-functional air purification filter 325 (e.g., the multi-functional air purification filter 100a, 100b, or 100c of FIGS. 1A to 1C), and a blowing fan 331. Since the multi-functional air purification filter 325 includes a photocatalytic material, the purification device 300 may further include a light source 329. The light source 301 may be disposed or configured to radiate light to the multi-functional air purification filter 325, inside the housing 301. In some embodiments, the purification device 300 may include a controller 335 for performing the operation for driving the blowing fan 331 and/or radiation from the light source 329 (e.g., recycling of the multi-functional air purification filter 325) and may include a sensor 333 for detecting the air quality inside the purification device 300.

According to various embodiments, the pre-filter 321 may be a component for filtering out relatively large dust particles, among floating materials or foreign bodies in the air and may be disposed closest to the inlet 313. The HEPA filter 323 may be a component disposed behind the pre-filter 321 to filter, e.g., fine dust which is not filtered by the pre-filter 321. In some embodiments, the support layer 110a or 110b and/or the filter layer 130a or 130b of the multi-functional air purification filter 325 may have substantially the same material or structure as the HEPA filter 323. According to an embodiment, the pre-filter 321 may primarily filter dust, and the HEPA filter 323 having relatively higher performance than the pre-filter 321 may secondarily filter dust. Here, the HEPA filter 323 may be formed of, e.g., glass fiber. Although not shown in the drawings, a deodorizing filter including activated carbon may be further included between the pre-filter 321 and the HEPA filter 323 or behind the HEPA filter 323. The filters may be arranged in the order shown in FIG. 4 or may be arranged in a different order. According to an embodiment, at least one of the pre-filter 321 and the HEPA filter 323 may be omitted.

According to various embodiments, the light source 329 may radiate light toward the multi-functional air purification filter 325 including a photocatalytic material. The photocatalytic material, e.g., titanium dioxide or zinc oxide, of the multi-functional air purification filter 325 may react with the light radiated from the light source 329 to generate electron-hole pairs, and it may reduce Cu(II) produced by oxidation reaction with virus to Cu(I). According to an embodiment, the light source 329 may be implemented as a device, such as a fluorescent lamp or an incandescent lamp or an LED, and it may output at least one type of light among white light, red light, green light, blue light, ultraviolet light, visible light, or infrared light. In another embodiment, the light source 329 may be provided in the form of an assembly with a lens assembly, such as a Fresnel lens, a convex lens, or a concave lens. According to an embodiment, at least one parameter among the brightness, temperature, color, light focusing, light emission timing, and light emission direction of the light source 329 may be controlled by the controller 335.

According to various embodiments, the light source 329 may radiate light along the direction of the flow of air inside the housing 301, and the light radiated from the light source 329 may reach the multi-functional air purification filter 325. In another embodiment, the light source 329 may radiate light in a direction opposite to the flow direction of the air, thereby causing a photocatalytic reaction in the multi-functional air purification filter 325. According to an embodiment, the direction in which light is radiated to the multi-functional air purification filter 325 may be variously selected or combined.

According to various embodiments, the blowing fan 331 may introduce air from the outside of the purification device 300 into the housing 301 through the inlet 313. The air sucked in by the blowing fan 331 may be purified while passing through various filters (pre-filter 321, HEPA filter 323, and multi-functional air purification filter 325) and be discharged to the outside through the outlets 315a and 315b. The blowing fan 331 may be operated under the control of the controller 335 and may control the flow of air under the control of the controller 335.

According to various embodiments, the sensor 333 may be a sensor that measures the quality of air inside the purification device 300. For example, the sensor 333 may measure the type and concentration of a substance included in the air. In some embodiments, the sensor 333 may be disposed in an inner space of the purification device 300, e.g., in a position adjacent to the outlets 315a and 315b of the purification device 300. Alternatively, the sensor 333 may be disposed in a position adjacent to the multi-functional air purification filter 325 in the inner space of the purification device 300.

According to various embodiments, the controller 335 may control the overall operation of the purification device 300, and for example, may control the operation of the light source 329 and the blowing fan 331. According to various embodiments, the controller 335 may control the light source 329 and/or the blowing fan 331 based on the air quality information provided from the sensor 333 and an external electronic device (e.g., a smart phone or a smart home hub). The controller 335 may be a processor (e.g., a microprocessor or central processing unit (CPU)) that may execute, for example, a program (software) to control at least one other component (e.g., a hardware or software component) of the purification device 300 coupled with the controller 335, and may perform various data processing or computation. According to one embodiment, as at least part of the data processing or computation, the controller 335 (or processor) may load a command or data received from another component (e.g., the sensor or communication module) onto a volatile memory, process the command or the data stored in the volatile memory, and store resulting data in a non-volatile memory.

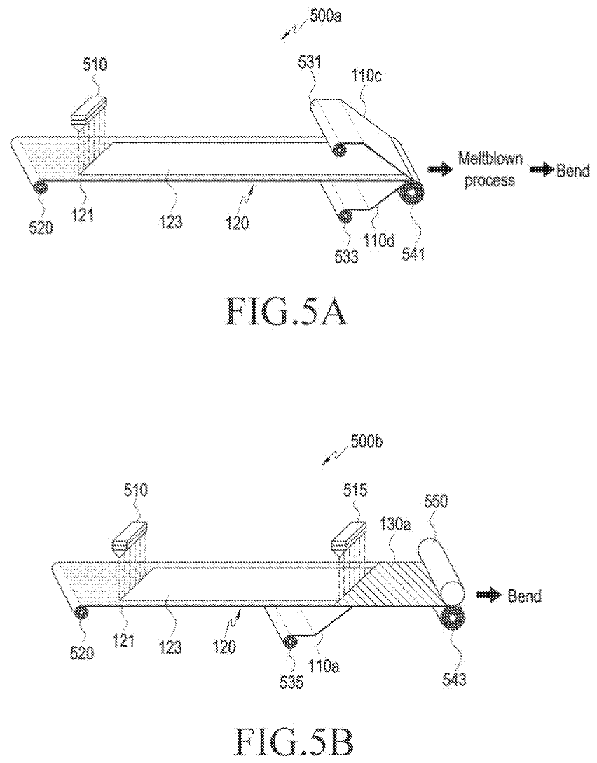

FIGS. 5A and 5B are views illustrating manufacturing devices of a multi-functional air purification filter according to various embodiments.

Referring to FIG. 5A, a manufacturing device 500a may include a coating device 510, a mesh layer roll 520, a first support layer roll 531, a second support layer roll 533, and a support layer/mesh layer bonding roll 541.

The mesh body 121 of the mesh layer 120 may be wound around the mesh layer roll 520, and the mesh body 121 released from the mesh layer roll 520 may be conveyed to the support layer/mesh layer bonding roll 541.

The coating device 510 may coat a hybrid material on the surface of the mesh body 121. The hybrid material may include a photocatalytic material and an adsorption material (and a sterilizing material). According to an embodiment, the coating layer 123 formed on the surface of the mesh body 121 may be dried, e.g., through hot air.

A first support layer 110c may be wound around the first support layer roll 531, and the first support layer 110c released from the first support layer roll 531 may be conveyed to the support layer/mesh layer bonding roll 541. The first support layer 110c may be disposed on the coating layer 123 that is a surface of the mesh layer 120. According to an embodiment, the first support layer 110c may be coupled, coated, or attached (an adhesive may be used) on the coating layer 123 that is a surface of the mesh layer 120.

A second support layer 110d may be wound around the second support layer roll 533, and the second support layer 110d released from the second support layer roll 533 may be conveyed to the support layer/mesh layer bonding roll 541. The second support layer 110d may be disposed on another surface of the mesh layer 120 (or the surface opposite to the surface of the mesh layer 120). According to an embodiment, the second support layer 110d may be coupled, coated, or attached (an adhesive may be used) on the other surface of the mesh layer 120.

A combination/assembly of the mesh layer 120, the first support layer 110c, and the second support layer 110d may be wound on the support layer/mesh layer bonding roll 541.

Thereafter, a melt-blown process for forming a filter layer 130a and a bending process for forming a multi-functional air purification filter in a zig-zag shape may be performed on the combination/assembly of the mesh layer 120, the first support layer 110c, and the second support layer 110d, released from the support layer/mesh layer bonding roll 541.

According to various embodiments, a pressing roll may be disposed to face the support layer/mesh layer bonding roll 541 to press the mesh layer 120, the first support layer 110c, and the second support layer 110d at a high temperature (50° C. to 150° C.) to increase the inter-layer bonding force.

Referring to FIG. 5B, a manufacturing device 500b may include a coating device 510, a melt blowing device 515, a mesh layer roll 520, a support layer roll 535, a filter layer/support layer/mesh layer bonding roll 543, and a pressing roll 550.

The mesh body 121 of the mesh layer 120 may be wound around the mesh layer roll 520, and the mesh body 121 released from the mesh layer roll 520 may be conveyed to the filter layer/support layer/mesh layer bonding roll 543.

The coating device 510 may coat a hybrid material on the surface of the mesh body 121. The hybrid material may include a photocatalytic material and an adsorption material (and a sterilizing material). According to an embodiment, the coating layer 123 formed on the surface of the mesh body 121 may be dried, e.g., through hot air.

The melt blowing device 515 may coat a polymer (plastic) material (PET, PP, PE, PTFE, etc.) on the coating layer 123, which is a surface of the mesh layer 120. The melt blowing device 515 may perform a melt blown process that fuses a polymer (plastic) material and extrudes it through a nozzle. The filter layer 130a may be disposed on the coating layer 123 that is a surface of the mesh layer 120 by the melt blowing device 515.

A support layer 110a may be wound around the support layer roll 535, and the support layer 110a released from the support layer roll 535 may be conveyed to the filter layer/support layer/mesh layer bonding roll 543. The support layer 110a may be disposed on another surface of the mesh layer 120 (or the surface opposite to the surface of the mesh layer 120). According to an embodiment, the support layer 110a may be coupled, coated, or attached (an adhesive may be used) on the other surface of the mesh layer 120.

The pressing roll 550 may be disposed to face the filter layer/support layer/mesh layer bonding roll 543 and may press the filter layer 130a, the mesh layer 120, and the support layer 110a at a high temperature (50° C. to 150° C.) to increase the inter-layer bonding force.

The pressed combination/assembly of the filter layer 130a, the mesh layer 120, and the support layer 110a may be wound on the filter layer/support layer/mesh layer bonding roll 543.

Thereafter, a bending process for forming a zig-zagged multi-functional air purification filter may be formed on the combination/assembly of the filter layer 130a, the mesh layer 120, and the support layer 110a, released from the filter layer/support layer/mesh layer bonding roll 543.

According to various embodiments, a multi-functional air purification filter may comprise a breathable support layer, a mesh layer disposed on a surface of the support layer, and a filter layer disposed on a surface of the mesh layer. The multi-functional air purification filter at least partially has a shape with a plurality of bends. The mesh layer may include a coating layer of at least one of a photocatalytic material, an adsorption material, or a sterilizing material.

According to various embodiments, the photocatalytic material may include at least one of titanium dioxide (TiO₂), tungsten oxide (WO₃), zirconium oxide (ZrO₂), zinc oxide (ZnO), cadmium sulfide (CdS), or vanadium oxide (V₂O₃).

According to various embodiments, the adsorption material may include at least one of zeolite, sepiolite, mesoporous silica (mesoporous SiO₂), silica (SiO₂), activated carbon, or clay.

According to various embodiments, the sterilizing material may include at least one of Cu, a Cu compound, Ag, an Ag compound, Zn, or a Zn compound.

According to various embodiments, a ratio of the adsorption material to the photocatalytic material may be 1.0 wt % to 80.0 wt % or 5 wt % to 50 wt %.

According to various embodiments, the ratio of the sterilizing material to the photocatalytic material may be 0.1 wt % to 10.0 wt % or 0.1 wt % to 3 wt %.

According to various embodiments, the support layer may be formed of a material including at least one of nonwoven fabric, felt, polyethylene terephthalate (PET), or polypropylene (PP).

According to various embodiments, the mesh layer may include a body with a mesh structure and a coating layer including a photocatalytic material, an adsorption material, and a sterilizing material and disposed on a surface of the body.

According to various embodiments, the mesh layer may be formed of a material including at least one of polyethylene terephthalate (PET), polypropylene (PP), polyethylene (PE), polytetrafluoroethylene (PTFE), stainless use steel (SUS), Ti, Al, or Cu.

According to various embodiments, the filter layer may be formed of a material including at least one of PET, PP, PE, or PTFE.

According to various embodiments, the multi-functional air purification filter may further comprise beads filling an empty space provided by the shape with the plurality of bends of the multi-functional air purification filter and containing a photocatalytic material, an adsorption material, and a sterilizing material.

According to various embodiments, a purification device may comprise a multi-functional air purification filter including a breathable support layer, a mesh layer disposed on a surface of the support layer, and a filter layer disposed on a surface of the mesh layer. The multi-functional air purification filter may at least partially have a shape with a plurality of bends. At least one of the support layer, the mesh layer, or the filter layer may include a photocatalytic material, an adsorption material, or a sterilizing material.

According to various embodiments, the purification device may further comprise a housing receiving the multi-functional air purification filter and a blowing fan disposed in the housing and configured to introduce external air through the multi-functional air purification filter to an inside of the housing.

According to various embodiments, the purification device may further comprise a light source for radiating light to the multi-functional air purification filter.

While embodiments of the disclosure have been shown and described, it will be apparent to those of ordinary skill in the art that various changes in form and detail may be made thereto without departing from the spirit and scope of the disclosure as defined by the following claims.

What is claimed is:

1. A multi-functional air purification filter comprising:
a breathable support layer;
a mesh layer provided on the breathable support layer; and
a filter layer provided on the mesh layer,
wherein at least a portion of the multi-functional air purification filter has a plurality of bends, and
wherein the mesh layer comprises a coating layer of at least one of a photocatalytic material, an adsorption material, or a sterilizing material.

2. The multi-functional air purification filter of claim 1, wherein the photocatalytic material comprises at least one of titanium dioxide (TiO₂), tungsten oxide (WO₃), zirconium oxide (ZrO₂), zinc oxide (ZnO), cadmium sulfide (CdS), or vanadium oxide (V₂O₃).

3. The multi-functional air purification filter of claim 1, wherein the adsorption material comprises at least one of zeolite, sepiolite, mesoporous silica (mesoporous SiO₂), silica (SiO₂), activated carbon, or clay.

4. The multi-functional air purification filter of claim 1, wherein the sterilizing material comprises at least one of Cu, a Cu compound, Ag, an Ag compound, Zn, or a Zn compound.

5. The multi-functional air purification filter of claim 1, wherein a ratio of the adsorption material to the photocatalytic material is 1.0 wt % to 80.0 wt %.

6. The multi-functional air purification filter of claim 1, wherein a ratio of the sterilizing material to the photocatalytic material is 0.1 wt % to 10.0 wt %.

7. The multi-functional air purification filter of claim 1, wherein the breathable support layer comprises at least one of nonwoven fabric, felt, polyethylene terephthalate (PET), or polypropylene (PP).

8. The multi-functional air purification filter of claim 1, wherein the mesh layer comprises at least one of polyethylene terephthalate (PET), polypropylene (PP), polyethylene (PE), polytetrafluoroethylene (PTFE), stainless use steel (SUS), Ti, Al, or Cu.

9. The multi-functional air purification filter of claim 1, wherein the filter layer comprises at least one of polyethylene terephthalate (PET), polypropylene (PP), polyethylene (PE), or polytetrafluoroethylene (PTFE).

10. The multi-functional air purification filter of claim 1, further comprising beads filling empty spaces provided by the plurality of bends of the multi-functional air purification filter, the beads comprising a photocatalytic material, an adsorption material, and a sterilizing material.

11. A purification device comprising:
a multi-functional air purification filter comprising a breathable support layer, a mesh layer provided on the breathable support layer, and a filter layer provided the mesh layer,
wherein at least a portion of the multi-functional air purification filter has a plurality of bends, and
wherein the mesh layer comprises a coating layer of at least one of a photocatalytic material, an adsorption material, or a sterilizing material.

12. The purification device of claim 11, further comprising:
a housing in which the multi-functional air purification filter is provided; and
a blowing fan provided in the housing and configured to introduce external air through the multi-functional air purification filter to an inside of the housing.

13. The purification device of claim 11, further comprising a light source configured to irradiate the multi-functional air purification filter with light.

14. The purification device of claim 11, wherein the photocatalytic material comprises at least one of titanium dioxide (TiO₂), tungsten oxide (WO₃), zirconium oxide (ZrO₂), zinc oxide (ZnO), cadmium sulfide (CdS), or vanadium oxide (V₂O₃),
wherein the adsorption material comprises at least one of zeolite, sepiolite, mesoporous silica (mesoporous SiO₂), silica (SiO₂), activated carbon, or clay, and
wherein the sterilizing material comprises at least one of Cu, a Cu compound, Ag, an Ag compound, Zn, or a Zn compound.

15. The purification device of claim 11, wherein a ratio of the adsorption material to the photocatalytic material is 1.0 wt % to 80.0 wt %.

16. The purification device of claim 11, wherein a ratio of the sterilizing material to the photocatalytic material is 0.1 wt % to 10.0 wt %.

17. The purification device of claim 11, wherein the breathable support layer comprises at least one of nonwoven fabric, felt, polyethylene terephthalate (PET), or polypropylene (PP).

18. The purification device of claim 11, wherein the mesh layer comprises at least one of polyethylene terephthalate (PET), polypropylene (PP), polyethylene (PE), polytetrafluoroethylene (PTFE), stainless use steel (SUS), Ti, Al, or Cu.

19. The purification device of claim 11, wherein the filter layer comprises at least one of polyethylene terephthalate (PET), polypropylene (PP), polyethylene (PE), or polytetrafluoroethylene (PTFE).

20. The purification device of claim 11, further comprising beads filling an empty spaces provided by the plurality of bends of the multi-functional air purification filter, the beads comprising a photocatalytic material, an adsorption material, and a sterilizing material.
